(19) United States
(12) Patent Application Publication
(10) Pub. No.: US 2008/0300253 A1
(43) Pub. Date: Dec. 4, 2008

(54) TREATMENT OF INFLAMMATORY DISORDERS WITH PRAZIQUANTEL

(75) Inventors: Deug Yong Shin, Choongnam (KR); Tae Saeng Choi, Choongnam (KR); Dong Hoon Jin, Seoul (KR); Min Kyung Cho, Kyungnam (KR)

Correspondence Address: KNOBBE MARTENS OLSON & BEAR LLP, 2040 MAIN STREET, FOURTEENTH FLOOR, IRVINE, CA, 92614, US

(73) Assignee: Gencross, Inc., Choongnam (KR)

(21) Appl. No.: 12/123,268

(22) Filed: May 19, 2008

Related U.S. Application Data
(63) Continuation of application No. 11/333,836, filed on Jan. 17, 2006, now abandoned.

(30) Foreign Application Priority Data
Jan. 18, 2005 (KR) 10-2005-0004490

(51) Int. Cl. A61K 31/4985; A61P 19/02; A61P 31/00; A61P 25/00; A61P 9/00; A61P 11/00

(52) U.S. Cl. 514/250

(57) ABSTRACT

Methods of treating and/or preventing disorders mediated by one or more of TNF-α, NF-κB, IKK-α, IKK-β, ATF-2 and p38 kinase by administration of praziquantel, or a pharmaceutically acceptable salt, prodrug, ester or amide thereof. These disorders include inflammatory disorders such as autoimmune diseases.

### CROSS-REFERENCE TO RELATED APPLICATIONS

This application is a continuation of U.S. patent application Ser. No. 11/333,836, filed Jan. 17, 2006, which claims priority under 35 U.S.C. § 119(a) to Korean Patent Application No. 10-2005-0004490, filed Jan. 18, 2005 in the name of Deug Yong Shin et al. entitled “A Pharmaceutical Composition Comprising Praziquantel, a Derivative Thereof, and Salt Thereof”, the entire contents of which are expressly incorporated herein by reference, including any drawings.

### FIELD OF THE INVENTION

The present invention relates to a method for inhibiting the amount and/or activity of TNF-α, NF-κB, IKK-α, IKK-β, ATF-2 or p38 kinase by treatment with praziquantel. More specifically, the invention relates to the use of praziquantel for treating or preventing a disease resulting from increased amounts and/or activity of one or more of these proteins, including various inflammatory disorders.

### BACKGROUND OF THE INVENTION

Inflammation is a process which occurs as a response to various infectious agents and/or toxins. One of the major molecular pathways which promotes inflammation involves the activation (phosphorylation) of p38 kinase which results in activation of the pro-inflammatory cytokine TNF-α, either directly or indirectly, via IKK, IκB and NFκB. TNF-α, NF-κB, IKK-α, IKK-β, ATF-2 and p38 kinase are mediators of various inflammatory diseases, infectious diseases, immune diseases and malignant diseases. Because of their involvement in inflammation, these molecules have long been targets for novel drug development.

The eukaryotic nuclear factor κB (NF-κB) is a pleiotropic transcriptional activator in various inflammatory and disease states, and also regulates cytokine levels, including TNF-α. NF-κB promotes inflammation by enhancing production of TNF-α, protects cancer cells from apoptotic cell death, and may enhance their growth activity. In addition, NF-κB functions as an HIV transcriptional activator. The activity of NF-κB is tightly regulated by its interaction with inhibitory IκB proteins. In most resting cells, NF-κB is sequestered in the cytoplasm in an inactive form associated with inhibitory molecules such as IκBα and IκBβ. This interaction blocks the ability of NF-κB to bind to DNA and results in the NF-κB complex being primarily localized to the cytoplasm. Activation of the NF-κB signaling cascade in response to various inducers, such as bacterial or viral toxins, results in degradation of IκB which allows translocation of NF-κB to the nucleus where it binds to the enhancer or promoter regions of target genes and regulates their transcription.

The activation of NF-κB by extracellular inducers depends on the phosphorylation and subsequent degradation of IκB proteins. Activation of NF-κB is achieved through the action of IKK protein kinase which contains two catalytic subunits (IKKα and IKKβ). IKKα and IKKβ proteins phosphorylate IκB and NF-κB. These phosphorylation events result in rapid degradation of IκB.

Activating transcription factor-2 (ATF-2) is the substrate protein of p38 kinase and is activated via phosphorylation by activated (phosphorylated) p38 kinase. The net result of this pathway is production of TNF-α, one of the major pro-inflammatory cytokines.

TNF-α is released by monocytic phagocytes in response to various immunostimulators, including lipopolysaccharide (LPS). LPS is an integral part of the outer cell membrane of gram-negative bacteria which is released after infection and activates inflammatory pathways. Administration of TNF-α results in inflammation, bleeding, coagulation and acute phase reactions in infection and shock. Excessive TNF-α production is a hallmark of many inflammatory diseases, including arthritis, graft versus host disease (GVHD), cerebral malaria, chronic lung inflammatory diseases and reperfusion injuries. TNF-α also functions as a mediator of tissue damage in myocardial infarction, cerebral apoplexy and circulatory shock in their initial inflammatory stages.

TNF-α also functions as an activator for replication of retroviruses, including HIV-1. Acquired Immune Deficiency Syndrome (AIDS) results from infection of T-lymphocytes by the human immunodeficiency virus (HIV). There are three types of HIV: HIV-1, HIV-2 and HIV-3. HIV impairs the T cell-mediated immune system, resulting in opportunistic infections. HIV-1 and HIV-2 infect T-lymphocytes only after T-cells become active since the expression and replication of viral proteins is mediated and maintained via T-cell activation. Even after activated T-cells are infected with HIV, T-lymphocytes must remain active for HIV gene expression and/or replication to occur. By keeping T-lymphocytes active, cytokines, particularly TNF-α, are associated with HIV protein expression and/or virus replication mediated by T-cells. Thus, maintenance of T-lymphocytes by HIV can be abrogated in an infected individual if TNF-α activity is inhibited. Cytokines such as TNF-α also activate HIV replication in monocytes and/or macrophages. Thus, inhibition of such cytokine activity would prevent T-lymphocyte activation.

Although TNF-α inhibitors are being developed, they suffer from serious side effects, including vomiting, nausea, dizziness, adrenal gland atrophy and suppressed hormone production. Thus, there is a need for a safe and effective inhibitor of TNF-α production.

### SUMMARY OF THE INVENTION

The present invention provides a method for treating a disorder involving an increased amount and/or activity of NF-κB, TNF-α, IKK-α, IKK-β, p38 kinase or ATF-2, comprising identifying a mammal in need of such treatment; and administering praziquantel, or a pharmaceutically acceptable salt, prodrug, ester or amide thereof, to the mammal. In one embodiment, the disorder is an inflammatory disorder. In one aspect of this embodiment, the inflammatory disorder is an autoimmune disorder. In yet another embodiment, the disorder is rheumatoid arthritis, sepsis, septic shock, bronchitis, rheumatoid spondylitis, diabetes, asthma, alopecia, toxic-resistance shock, reperfusion lesion, malaria, meningitis, psoriasis, hemorrhagic heart failure, fibrosis disease, acute inflammation, oncosis, autoimmune disease, AIDS, HIV infection, osteoarthritis, arthritis, chronic laryngostasis, Crohn's disease, ulcerative colitis, hepatitis, hemorrhagic shock, multicentric sclerosis, radiation lesion and oxygen luxus lesion, allergic rhinitis, dermatitis, depression, gnathostatic syndrome, brain infarct, epileptogenic pneumonia, myocardial infarction, inflammatory disease, arteriosclerosis, hypertension, cardiovascular disease or lupus. In another embodiment, the mammal is a human.

The present invention also provides a method for inhibiting the production and/or activity of TNF-α in a mammal in need thereof, comprising identifying a mammal in need of such treatment, and administering praziquantel, or a pharmaceutically acceptable salt, prodrug, ester or amide thereof, to the mammal. In one embodiment, the mammal is a human. In another embodiment, the TNF-α production and/or activity is associated with an inflammatory disorder. In yet another embodiment, the inflammatory disorder is rheumatoid arthritis, sepsis, septic shock, bronchitis, rheumatoid spondylitis, diabetes, asthma, alopecia, toxic-resistance shock, reperfusion lesion, malaria, meningitis, psoriasis, hemorrhagic heart failure, fibrosis disease, acute inflammation, oncosis, autoimmune disease, AIDS, HIV infection, osteoarthritis, arthritis, chronic laryngostasis, Crohn's disease, ulcerative colitis, hepatitis, hemorrhagic shock, multicentric sclerosis, radiation lesion and oxygen luxus lesion, allergic rhinitis, dermatitis, depression, gnathostatic syndrome, brain infarct, epileptogenic pneumonia, myocardial infarction, inflammatory disease, arteriosclerosis, hypertension, cardiovascular disease or lupus.

Another embodiment of the present invention is a method of treating arthritis in a mammal in need thereof, comprising identifying a mammal in need of such treatment and administering praziquantel, or a pharmaceutically acceptable salt, prodrug, ester or amide thereof, to said mammal. In one aspect of this embodiment, the mammal is a human.

### BRIEF DESCRIPTION OF THE DRAWINGS

FIG. 1 shows the inhibitory activity of praziquantel on TNF-α synthesis, p38 kinase activity, ATF-2 activity, NFκB activity, IKK-α/β activity and IκB activity in a THP-1 immune cell line.

FIG. 2 shows the inhibitory activity of praziquantel on TNF-α synthesis, p38 kinase activity, ATF-2 activity, NFκB activity, IKK-α/β activity and IκB activity in a mouse treated with LPS/D-galactosamine.

FIG. 3 is a bar graph showing the amount of TNF-α in the blood when different amounts (12.5 mg/kg, 25 mg/kg or 50 mg/kg) of praziquantel are administered to mice prior to treatment with LPS/D-galactosamine.

### DETAILED DESCRIPTION OF THE PREFERRED EMBODIMENT

The present invention relates to the use of praziquantel, or a pharmaceutically acceptable salt, prodrug, ester or amide thereof, for inhibiting the production and/or activity of one or more of the following proteins: NF-κB, TNF-α, IKK-α, IKK-β, p38 kinase and ATF-2, and for treatment of disorders or diseases involving elevated amounts and/or activities of one or more of these proteins. Such disorders often involve inflammation mediated by one or more of these proteins, particularly TNF-α. Thus, praziquantel can be used to treat a range of inflammatory disorders.

Praziquantel is a compound having the following structure:

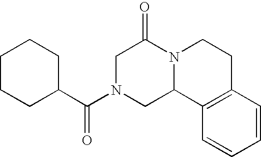

It is also known by its chemical name, 2-(cyclohexylcarbonyl)-1,2,3,6,7,11β-hexahydro-4H-pyrazino[2,1-α]isoquinoline-4-one.

Praziquantel is an antiparasitic drug that is used to treat the parasitic disease schistosomiasis (Cioli et al., Parasitology Res. 90:S3-S9, 2002). There has been no report of significant side effects of this drug during its 20 years of use as an antiparasitic agent. The toxicity of this compound is very low, both in acute and long-term experiments. The LD50 in rats is 2,000-3,000 mg/kg. No adverse effects were observed in dogs or cats dosed with 100 mg/kg.

Praziquantel exerts its anti-inflammatory effects by inhibiting the production and/or activity of one or more of NF-κB, TNF-α, IKK-α, IKK-β, p38 kinase and ATF-2. Praziquantel, or a pharmaceutically acceptable salt, prodrug, ester or amide thereof, can be used to prevent or treat diseases including, but not limited to, sepsis, septic shock, bronchitis, rheumatoid arthritis, rheumatoid spondylitis, diabetes, asthma, alopecia, toxic-resistance shock, reperfusion lesion, malaria, meningitis, psoriasis, hemorrhagic heart failure, fibrosis disease, acute inflammation, oncosis, autoimmune disease, AIDS, HIV infection, osteoarthritis, arthritis, chronic laryngostasis, Crohn's disease, ulcerative colitis, hepatitis, hemorrhagic shock, multicentric sclerosis, radiation lesion and oxygen luxus lesion, allergic rhinitis, dermatitis, depression, gnathostatic syndrome, brain infarct, epileptogenic pneumonia, myocardial infarction, inflammatory disease, arteriosclerosis, hypertension, cardiovascular disease and lupus.

Praziquantel may be used to treat a variety of vertebrates such as birds and mammals. Mammals suitable for treatment using the compositions and methods described herein include humans, primates, dogs, cats, rabbits, guinea pigs, horses, pigs, cows, and the like. A mammal having an inflammatory disorder, or a disorder involving increased production and/or activity of one or more of NF-κB, TNF-α, IKK-α, IKK-β, p38 kinase and ATF-2 is identified, followed by administration of a pharmaceutical composition comprising praziquantel, or a pharmaceutically acceptable salt, prodrug, ester or amide thereof. In one embodiment, the disorder has an inflammatory component. In another embodiment, the disorder is selected from the list provided above.

The term “pharmaceutical composition” refers to a mixture of praziquantel, or a pharmaceutically acceptable salt, prodrug, ester or amide thereof, with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glutamine, tris(hydroxymethyl)methylamine, and salts with amino acids such as arginine, lysine, and the like.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

The term “metabolite” refers to a compound to which praziquantel is converted within the cells of a mammal. The pharmaceutical compositions of the present invention may include a metabolite of praziquantel instead of praziquantel. The scope of the methods of the present invention includes those instances where praziquantel is administered to the patient, yet the metabolite is the bioactive entity.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

In a further aspect, the present invention relates to a method of treating a patient with a pharmaceutical composition as described herein.

The term “treating” or “treatment” does not necessarily mean total cure. Any alleviation of any undesired signs or symptoms of the disease to any extent or the slowing down of the progress of the disease can be considered treatment. Furthermore, treatment may include acts that may worsen the patient's overall feeling of well being or appearance. Treatment may also include lengthening the life of the patient, even if the symptoms are not alleviated, the disease conditions are not ameliorated, or the patient's overall feeling of well being is not improved.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, rectal, topical, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly in the renal or cardiac area, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions of the present invention may be manufactured in a manner that is itself known, e.g., by means of conventional mixing, dissolving, granulating, dragee-making, levigating, emulsifying, encapsulating, entrapping or tabletting processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

For topical administration, the compounds may be formulated for administration to the epidermis as ointments, gels, creams, pastes, salves, gels, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally, including sublingually, which include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions of the present invention can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.5 to 1000 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the present invention will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 6000 mg of each ingredient, preferably between 1 mg and 5000 mg, e.g. 25 to 5000 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions of the present invention or a pharmaceutically acceptable salt thereof calculated as the free base, the composition being administered 1 to 4 times per day. Alternatively the compositions of the invention may be administered by continuous intravenous infusion, preferably at a dose of each ingredient up to 400 mg per day. Thus, the total daily dosage by oral administration of each ingredient will typically be in the range 1 to 2500 mg and the total daily dosage by parenteral administration will typically be in the range 0.1 to 400 mg. Suitably the compounds will be administered for a period of continuous therapy, for example for a week or more, or for months or years.

In one embodiment, the dose of the pharmaceutical composition comprising praziquantel or a pharmaceutically acceptable salt, prodrug, ester or amide thereof, is from about 10 to about 50 mg per day.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen that maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

The compositions described herein may also be used in the preparation of a medicament for treatment of any of the disorders described above.

### Example 1

### Preparation of the Sample

Praziquantel was obtained from SIGMA Aldrich (cat #p4668) and dissolved in triple distilled water for use in the studies described below.

### Example 2

### Inhibition of NF-κB Activity and TNF-α Synthesis in THP-1 Cells

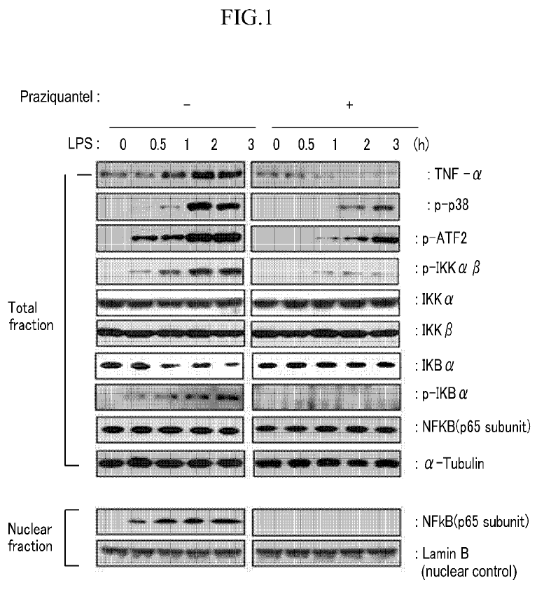

The THP-1 human monocytic cell line was treated with 200 nM phorbol myristate acetate (PMA) for 24 hours, resulting in differentiation into functional macrophages. Differentiated THP-1 cells were treated with 2 μg/ml lipopolysaccharide (LPS), and total proteins or nuclear proteins were isolated 0.5, 1, 2, and 3 hours later. In some differentiated THP-1 cell cultures, a combination of LPS and 100 nM praziquantel was added prior to isolation of total and nuclear proteins at the same time points. Both total protein and nuclear protein were quantitated by Western blotting. As shown in FIG. 1, there was a significant increase in the amount of TNF-α one, two and three hours after LPS treatment. In contrast, much less TNF-α was observed at these time points in THP-1 cells treated with LPS and praziquantel. In fact, at the two and three hour time points, TNF-α was barely detectable in THP-1 cells treated with both LPS and praziquantel. Significant reductions in the active (phosphorylated) forms of p38, ATF-2 and IKK α/β were also observed in THP-1 cells treated with LPS and praziquantel compared to LPS alone (FIG. 1). Total NF-κB levels did not change in the presence of LPS, and were not affected by praziquantel; however, translocation of this transcription factor to the nucleus did not occur in the presence of praziquantel since NF-κB was absent in the nuclear fraction (FIG. 1). Thus, praziquantel inhibits LPS-induced NF-κB activity by preventing its translocation to the nucleus where it exerts its transcriptional activation effects. Neither α-tubulin nor lamin B protein was affected by either LPS or praziquantel treatment (FIG. 1).

### Example 3

### In Vivo Reduction of TNF-α Levels by Praziquantel

An acute mouse in vivo sepsis model was used to determine the ability of praziquantel to reduce TNF-α levels. C57BL/6 mice were used which were 7 to 8 weeks old and weighed 25 to 30 g. Fodder (Daehan Biolink) and water were administered ad libitum, and the temperature and relative humidity of the nursery cage were maintained at 21-24° C. and 40-80%, respectively. In addition, the nursery cage light was controlled to switch between day and night every 12 hours. Eight to ten mice were assigned to each experimental group. Each mouse was intraperitoneally (ip) administered 8 ng LPS (Sigma Aldrich) and 8 mg D-galactosamine (SIGMA), each dissolved in 100 μl distilled water, to induce acute sepsis. In order to investigate the effect of praziquantel on TNF-α levels in blood, praziquantel was orally administered to each experimental group at dosages of 12.5 mg/kg, 25 mg/kg or 50 mg/kg, 30 minutes prior to treatment with LPS/D-galactosamine. The blood was sampled 1, 3 and 5 hours after treatment with praziquantel. Serum was isolated from the sampled blood, and the amount of TNF-α was measured by ELISA (R&D Systems, Minneapolis, Minn.).

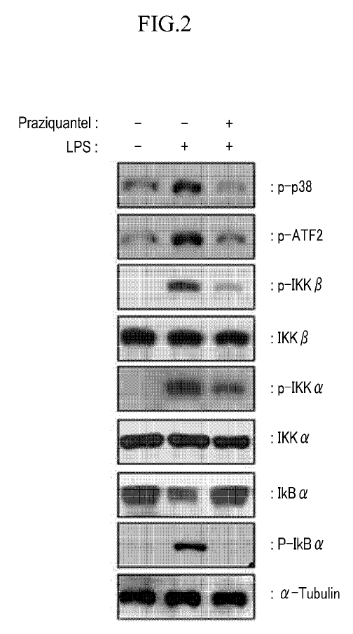

In the presence of LPS/D-galactosamine and absence of praziquantel, there was a large increase in the amount of TNF-α to about 6 times the control amount after 5 hours (FIG. 2). Pre-treatment of mice with praziquantel resulted in a dose-dependent decrease in the amount of TNF-α in blood serum. Thus, praziquantel significantly inhibits LPS/D-galactosamine-induced production of TNF-α in vivo.

### Example 4

### Enhancement of Survival of LPS/D-Galactosamine-Treated Mice by Praziquantel

Female C57BL6 mice (8-10 per experimental group) were treated as described above to induce acute sepsis, and the same doses of praziquantel were administered 30 minutes prior to treatment with LPS/D-galactosamine. The experiment was repeated three times, and the survival rates are shown in Table 1.

TABLE 1

| Prazi- | | | | | | | | | | |
|---|---|---|---|---|---|---|---|---|---|---|
| Quantel | | | | | | | | | | |
| Treatment | Survival rate, % (# of mice) | | | | | | | | | |
| (mg/kg) | 0 h | 5 h | 6 h | 7 h | 8 h | 9 h | 10 h | 11 h | 12 h | >24 h |
| 0 | 100 | 100 | 73 | 7 | 7 | 7 | 7 | 7 | 7 | 3 |
| | (30/30) | (30/30) | (22/30) | (2/30) | (2/30) | (2/30) | (2/30) | (2/30) | (2/30) | (1/30) |
| 12.5 | 100 | 100 | 86 | 57 | 50 | 43 | 14 | 14 | 14 | 14 |
| | (30/30) | (30/30) | (26/30) | (17/30) | (15/30) | (13/30) | (4/30) | (4/30) | (4/30) | (4/30) |
| 25 | 100 | 100 | 93 | 71 | 71 | 71 | 71 | 71 | 64 | 64 |
| | (30/30) | (30/30) | (28/30) | (21/30) | (21/30) | (21/30) | (21/30) | (21/30) | (19/30) | (19/30) |
| 50 | 100 | 100 | 93 | 93 | 93 | 93 | 93 | 93 | 93 | 93 |
| | (30/30) | (30/30) | (28/30) | (28/30) | (28/30) | (28/30) | (28/30) | (28/30) | (28/30) | (28/30) |

The mice in each experimental group started dying 5 to 6 hours after induction of sepsis in three repetitive experiments, and 97% of the mice died in 24 hours, resulting in a 3% survival rate. In contrast, in mice that had received oral praziquantel 30 minutes prior to treatment with LPS/D-galactosamine exhibited a dramatic increase in survival rate over more than 24 hours (14% with 12.5 mg/kg; 64% with 25 mg/kg and 93% with 50 mg/kg). Thus, the increase in survival occurred in a dose-dependent manner.

### Example 5

### Inhibition of NF-κB, IκB, IKK and p38 Activity, and TNF-α Synthesis by Praziquantel

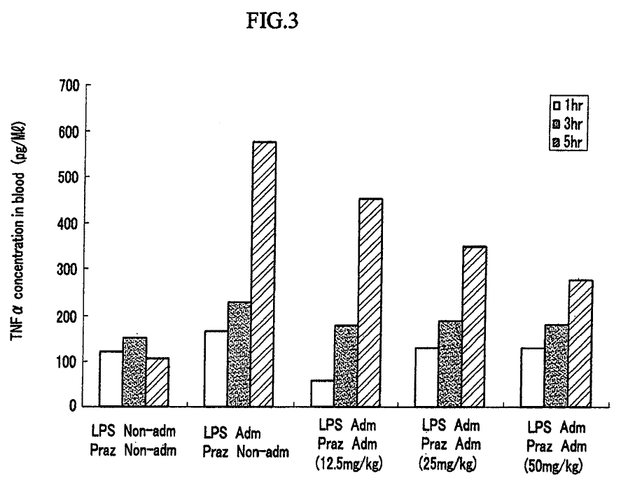

Mice were orally administered 50 mg/kg praziquantel 30 minutes prior to ip injection of 8 ng LPS and 8 mg D-galactosamine. Blood samples were taken 3 hours later, and mononuclear cells were isolated using Histopaque (Sigma Aldrich, cat #1080). Protein levels and their phosphorylation states were then determined by western blotting. As shown in FIG. 3, the phosphorylation of p38 kinase increased after treatment with LPS/D-galactosamine. Pre-treatment with praziquantel did not change the amount of p38 protein, but inhibited the phosphorylation of p38 kinase. p38 kinase is activated by phosphorylation, and also phosphorylates (and activates) ATF-2. Phosphorylation of ATF-2 increased after treatment with LPS/D-galactosamine, but was inhibited by pre-treatment with praziquantel (FIG. 3).

The amount of IKK-α and IKK-β proteins and their phosphorylation states were also examined. The amounts of these proteins did not change upon treatment with LPS/D-galactosamine; however; their phosphorylation increased. This phosphorylation was inhibited by pre-treatment with praziquantel. IKK-α and IKK-β are known to phosphorylate IκB, and phosphorylated IκB is degraded by a protease. The amount of IκB protein sharply decreased, and its phosphorylation increased, after treatment with LPS/D-galactosamine. Pre-treatment with praziquantel did not decrease the amount of IκB protein but inhibited its phosphorylation.

### Example 6

### Mouse Collagen-Induced Arthritis Model

Male DBA/1 mice, obtained from the Jackson Laboratory, are maintained in groups of three to five in polycarbonate cages and fed standard mouse chow and water ad libitum. The environment is made specifically pathogen-free. Mice are immunized with bovine type II collagen (CII), at 8 to 12 weeks of age, as described previously (Arthritis Rheum. 2002, 46:1109, 2002; J. Immunol., 5846, 2005). Briefly, CII is dissolved in 0.05N acetic acid at 2 mg/ml, and emulsified (1:1 ratio) with complete Freund's adjuvant (CFA) at 4° C. The mice receive 0.1 ml of the emulsion, containing 100 μg of CII, in the base of the tail as a primary immunization. Booster injections are given into the footpad with 50 μg of CII, similarly dissolved and emulsified with CFA (1:1), 14 days after the primary immunization. CIA develops as early as 3 wk, peaks at 5-7 wk, and thereafter spontaneously resolves at 10 wk. Mice are divided into two groups. One group is administered vehicle (saline), and the other group is administered an oral suspension of 50 mg/kg praziquantel in saline starting 2 weeks after the CII booster injection. Praziquantel administration is continued once or twice a day for three weeks after the initial administration.

TNF-α and p38 levels in blood are then assayed as described above, and the incidence and severity of arthritis in the two groups of mice are determined by visual inspection. The group administered praziquantel exhibits significantly reduced serum levels of TNF-α and p38, and greatly reduced severity of arthritis.

It will be understood by those of skill in the art that numerous and various modifications can be made without departing from the spirit of the present invention. Therefore, it should be clearly understood that the forms of the present invention are illustrative only and are not intended to limit the scope of the present invention.

All documents and other information sources cited above are hereby incorporated in their entirety by reference.

What is claimed is:

1. A method for treating a disorder involving an increased amount and/or activity of a protein selected from the group consisting NF-κB, TNF-α, IKK-α, IKK-β, p38 kinase and ATF-2, comprising identifying a mammal in need of such treatment; and administering praziquantel, or a pharmaceutically acceptable salt, prodrug, ester or amide thereof, to said mammal.

2. The method of claim 1, wherein said disorder is an inflammatory disorder.

3. The method of claim 1, wherein said inflammatory disorder is an autoimmune disorder.

4. The method of claim 1, wherein said disorder is selected from the group consisting of rheumatoid arthritis, bronchitis, rheumatoid spondylitis, diabetes, asthma, alopecia, toxic-resistance shock, reperfusion lesion, malaria, meningitis, psoriasis, hemorrhagic heart failure, fibrosis disease, acute inflammation, oncosis, autoimmune disease, AIDS, HIV infection, osteoarthritis, arthritis, chronic laryngostasis, Crohn's disease, ulcerative colitis, hepatitis, hemorrhagic shock, multicentric sclerosis, radiation lesion and oxygen luxus lesion, allergic rhinitis, dermatitis, depression, gnathostatic syndrome, brain infarct, epileptogenic pneumonia, myocardial infarction, inflammatory disease, arteriosclerosis, hypertension, cardiovascular disease and lupus.

5. The method of claim 1, wherein said mammal is a human.

6. A method for inhibiting production and/or activity of TNF-α in a mammal in need thereof, comprising identifying a mammal in need of such treatment, and administering praziquantel, or a pharmaceutically acceptable salt, prodrug, ester or amide thereof, to said mammal.

7. The method of claim 6, wherein said mammal is a human.

8. The method of claim 6, wherein said TNF-α production and/or activity is associated with an inflammatory disorder.

9. The method of claim 8, wherein said inflammatory disorder is selected from the group consisting of rheumatoid arthritis, bronchitis, rheumatoid spondylitis, diabetes, asthma, alopecia, toxic-resistance shock, reperfusion lesion, malaria, meningitis, psoriasis, hemorrhagic heart failure, fibrosis disease, acute inflammation, oncosis, autoimmune disease, AIDS, HIV infection, osteoarthritis, arthritis, chronic laryngostasis, Crohn's disease, ulcerative colitis, hepatitis, hemorrhagic shock, multicentric sclerosis, radiation lesion and oxygen luxus lesion, allergic rhinitis, dermatitis, depression, gnathostatic syndrome, brain infarct, epileptogenic pneumonia, myocardial infarction, inflammatory disease, arteriosclerosis, hypertension, cardiovascular disease and lupus.

10. A method of treating rheumatoid arthritis in a mammal in need thereof, comprising identifying a mammal in need of such treatment, and administering praziquantel, or a pharmaceutically acceptable salt, prodrug, ester or amide thereof, to said mammal.

11. The method of claim 10, wherein said mammal is a human.
